(19) United States
(12) Patent Application Publication
(10) Pub. No.: US 2011/0130656 A1
(43) Pub. Date: Jun. 2, 2011

(54) MICROWAVE IMAGE RECONSTRUCTION APPARATUS AND METHOD

(76) Inventors: Seong-Ho SON, Daejeon (KR); Hyuk-Je KIM, Daejeon (KR); Jong-Moon LEE, Chungbuk (KR); Soon-Ik JEON, Daejeon (KR); Pual M. MEANEY, Hanover, NH (US); Keith D. PAULSEN, Hanover, NH (US); Tomasz M. GRZEGORCZYK, Newton, MA (US)

(21) Appl. No.: 12/627,404

(22) Filed: Nov. 30, 2009

(51) Int. Cl. A61B 6/00

(52) U.S. Cl. 600/430

(57) ABSTRACT

Provided are microwave image reconstruction apparatus and method. An apparatus for diagnosing a breast cancer, includes: a plurality of antennas for inserting a breast and configured to transmit and receive the electromagnetic wave; an electromagnetic wave transceiver configured to receive phase and amplitude information of the electromagnetic wave; an initial distribution value provider configured to set an initial dielectric constant and conductivity distribution value for dielectric constant and conductivity of a breast of a patient and in the tank; a first image reconstruction unit configured to transform the amplitude information of the electromagnetic wave through log transform and to obtain a first image by calculating a phase and amplitude information value of electric field generated from the electromagnetic wave; and a second image reconstruction unit configured to transform the first image in a value in a complex number form and to transform values to a second image.

### BACKGROUND OF THE INVENTION

1. Field of the Invention

The present invention relates to a microwave image reconstruction apparatus and method; and, more particularly, to a microwave image reconstruction apparatus and method for diagnosing a breast cancer.

2. Description of Related Art

In general, a breast cancer is adenocarcinoma growing in a breast. It is the most common cancer in women. Many studies have been made to find medical diagnosis and treatments for the breast cancer. It has not been found what causes the breast cancer. The breast cancer is often found from women who are like to have western style foods, from women in an age group from 35 to 50, from women experiencing menarche in her early age, from single women with no experience of pregnancy, from women delivered a first child after an age of 30, and from women not giving breastfeeding. Particularly, when a woman has a close family member getting a breast cancer, she has a great chance to get a breast cancer too.

The breast cancer is a major cause increasing a death rate of women. If a breast cancer is found in it's a late stage, a breast of a patient is often removed to treat the breast cancer. Such a treatment of the breast cancer may give a great shock to a patient. Although the breast cancer is successfully cured, a large percentage of women having experience of a breast cancer are finally died due to a complication indirectly or directly related to the breast cancer. Therefore, it is important to diagnose a breast cancer at an early stage as well as preventing a breast cancer.

Lately, a far-infrared ray, an X-ray mammography, and a ultrasound wave have been generally used to diagnose a breast cancer. The far-infrared ray based breast cancer diagnosis method measures a temperature of an internal tissue of a body and identifies a tumor tissue from a normal tissue based on the measuring result. However, the far-infrared ray based breast cancer diagnosis method has a disadvantage of a low accuracy in diagnosis of a breast cancer. The X-ray mammography based breast cancer diagnosis method detects a tumor tissue by permeating an X-ray mammography into a body. Due to the X-ray, the X-ray mammography based breast cancer diagnosis method is harmful to a human body. Particularly, when an X-ray is transmitted through a human body, some of X-ray permanently remains in a human body. The biggest problem with X-ray mammography is the poor sensitivity and poor specificity. Part of this is due to low radiographic density contrast between the normal and malignant tissue. In addition, the fibroglandular tissue can easily obscure the tumor for women with denser breasts. The ultrasound wave based breast cancer diagnosis method diagnoses a breast cancer by reconstructing an internal image of a breast using a ultrasound wave. However, the ultrasound wave based breast cancer diagnosis method has a problem of low sharpness in the reconstructed internal image. It is difficult to detect a tumor tissue in a breast by examining the reconstructed internal image of a breast. Accordingly, the ultrasound wave based breast cancer diagnosis method has low diagnosis accuracy.

In order to overcome these problems of the conventional breast cancer diagnosis methods, an electromagnetic wave based breast cancer diagnosis apparatus was introduced. The electromagnetic wave based breast cancer diagnosis apparatus diagnoses a breast cancer by reconstructing a tomographic image by analyzing the back-diffraction and thru propagation of an electromagnetic wave. Such an electromagnetic wave based breast cancer diagnosis method according to the related art will be described with reference to FIG. 1.

FIG. 1 is a diagram illustrating a method for diagnosis of a breast cancer using an electromagnetic wave according to the related art. Such an electromagnetic wave based breast cancer diagnosis method was introduced in U.S. Patent Publication No. 20040077943.

An electromagnetic wave based breast cancer diagnosis apparatus shown in FIG. 1 includes a tank and 16 transceiver antennas 100. The 16 transceiver antennas 100 are disposed in the tank. The tank is filled with a predetermined liquid. The 16 transceiver antennas 100 (#1, #2, . . . , #16) are arranged in a circular form. Operation of the electromagnetic wave based breast cancer diagnosis apparatus according to the related art will be described hereinafter.

A breast of a patient is located among the 16 transceiver antennas #1, #2, . . . , and #16 arranged in a circle. At first, the first antenna #1 is controlled to transmit an electromagnetic wave 120, and the other 15 antennas #2, #3, . . . , and #16 are controlled to receive the scattered electromagnetic wave 120. Then, information about amplitudes and phases of the received electromagnetic waves 120 are collected from the 15 antennas #2, #3, . . . , and #16. After collecting the information, the second antenna #2 is controlled to transmit an electromagnetic wave 120, and the other 15 antennas #1, #3, #4, . . . , and #16 are controlled to receive scattered electromagnetic wave 120. Then, information about the amplitudes and the phases of received electromagnetic waves 120 are collected from the 15 antennas #1, #3, #4, . . . , and #16. Such operation steps repeat until the 16^{th} antenna is controlled to transmit an electromagnetic wave 120. After collecting the information about the amplitude and phase of the electromagnetic waves transmitted from all of the antennas, an image of internal breast is reconstructed based on dielectric constant/conductivity distribution using a predetermined image reconstruction algorithm. A doctor examines this reconstructed image to determine whether a tumor 115 is grown in a breast or not. Therefore, the performance of the image reconstruction algorithm based on the dielectric constant/conductivity distribution is very important.

A Levenberg-Marquardt (LM) algorithm and a Tikhonov (TK) algorithm have been widely known as an image reconstruction algorithm. Since these image reconstruction algorithms use a least square method, the LM algorithm and the TK algorithm have disadvantages of a slow reconstruction speed and a smooth image problem.

### SUMMARY OF THE INVENTION

An embodiment of the present invention is directed to providing a microwave image reconstruction apparatus and method, and more particularly, to providing a microwave image reconstruction apparatus and method for quickly obtaining an initial valid estimation value and having a stable image reconstruction performance in order to improve an image reconstruction performance.

Other objects and advantages of the present invention can be understood by the following description, and become apparent with reference to the embodiments of the present invention. Also, it is obvious to those skilled in the art to which the present invention pertains that the objects and advantages of the present invention can be realized by the means as claimed and combinations thereof.

In accordance with an aspect of the present invention, there is provided an apparatus for diagnosing a breast cancer using an electromagnetic wave, the apparatus includes: a plurality of antennas disposed in a tank for inserting a breast and configured to transmit and receive the electromagnetic wave; an electromagnetic wave transceiver configured to receive phase and amplitude information of the electromagnetic wave received through the plurality of antennas; an initial distribution value provider configured to set an initial dielectric constant and conductivity distribution value for dielectric constant and conductivity of a breast of a patient and in the tank; a first image reconstruction unit configured to transform the amplitude information of the electromagnetic wave through log transform and to obtain a first image by calculating a phase and amplitude information value of electric field generated from the electromagnetic wave; and a second image reconstruction unit configured to transform the first image in a value in a complex number form and to transform values, which make a difference in a phase and an intensity between the measured electric field and the calculated electric field to have a minimum distance, to a second image.

In accordance with another aspect of the present invention, there is provided a method for diagnosing a breast cancer using an electromagnetic wave, the method includes: inserting a breast in a tank and transmitting and receiving an electromagnetic wave using a plurality of antennas disposed in the tank; setting an initial dielectric constant and conductivity distribution value for dielectric constant of predetermined liquid and conductivity of the breast and in the tank; transforming amplitude information of the electromagnetic wave through Log Transformation and obtaining first image information by calculating a phase and amplitude information value of electric field generated from the electromagnetic wave through a predetermined image algorithm; and transforming the obtained first image information to values in a complex number form, and transforming values that makes a difference in a phase and an intensity between measured electric field from the antennas and the calculated electric field to have a minimum Euclidean distance to a second image.

In accordance with another aspect of the present invention, there is provided a method for diagnosing a breast cancer using an electromagnetic wave, the method includes: setting an initial dielectric constant and conductivity distribution value for dielectric constant and conductivity of a breast of a patient; obtaining phase and amplitude information of electric field generated from the electromagnetic wave received from a plurality of extractable antennas that transmit and receive the electromagnetic wave, transforming the obtained amplitude information of the electromagnetic wave through Log Transform, and obtaining estimated image information by processing the phase and amplitude information of the electric field through a predetermined image algorithm; obtaining a phase value and an intensity value of an electric field by transforming the obtained estimated image information into values in a complex number form; detecting a difference between a phase value and an intensity value of an electric field measured from a breast of a patient and the obtained phase value and intensity value of the electric field for said obtaining a phase value and intensity value; and providing the image information when the detected error is in a predetermined range.

### BRIEF DESCRIPTION OF THE DRAWINGS

FIG. 1 is a diagram illustrating a method for diagnosis of a breast cancer using an electromagnetic wave according to the related art.

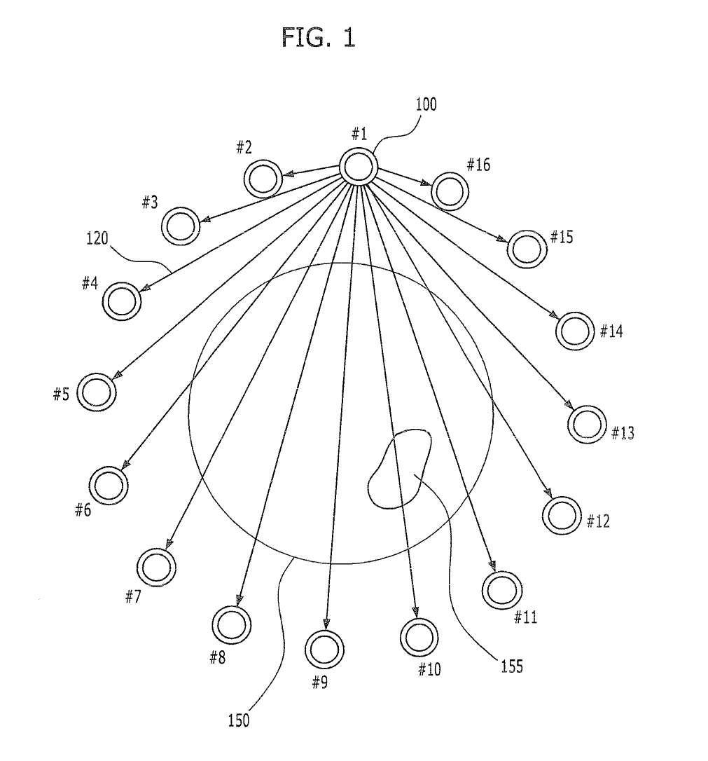

FIG. 2 is a perspective view of a breast cancer diagnosis apparatus in accordance with an embodiment of the present invention.

FIG. 3 is a flowchart illustrating two step microwave image reconstruction in accordance with another embodiment of the present invention.

FIG. 4 is a flowchart illustrating an image reconstruction algorithm in accordance with an embodiment of the present invention.

### DESCRIPTION OF SPECIFIC EMBODIMENTS

The advantages, features and aspects of the invention will become apparent from the following description of the embodiments with reference to the accompanying drawings, which is set forth hereinafter.

In the specification, a diagnosis apparatus and method according to embodiments of the present invention is exemplary described to diagnose a breast cancer. However, the present invention is not limited thereto. The diagnosis apparatus and method according to embodiments of the present invention can be applied to diagnose other cancers.

In the specification, a breast cancer diagnosis apparatus and method in accordance with an embodiment of the present invention will be described to use a microwave for diagnosing a breast cancer as an example of electromagnetic wave. However, the present invention is not limited thereto. The breast cancer diagnosis apparatus and method according to embodiments of the present invention can use any electromagnetic waves having various frequency bands.

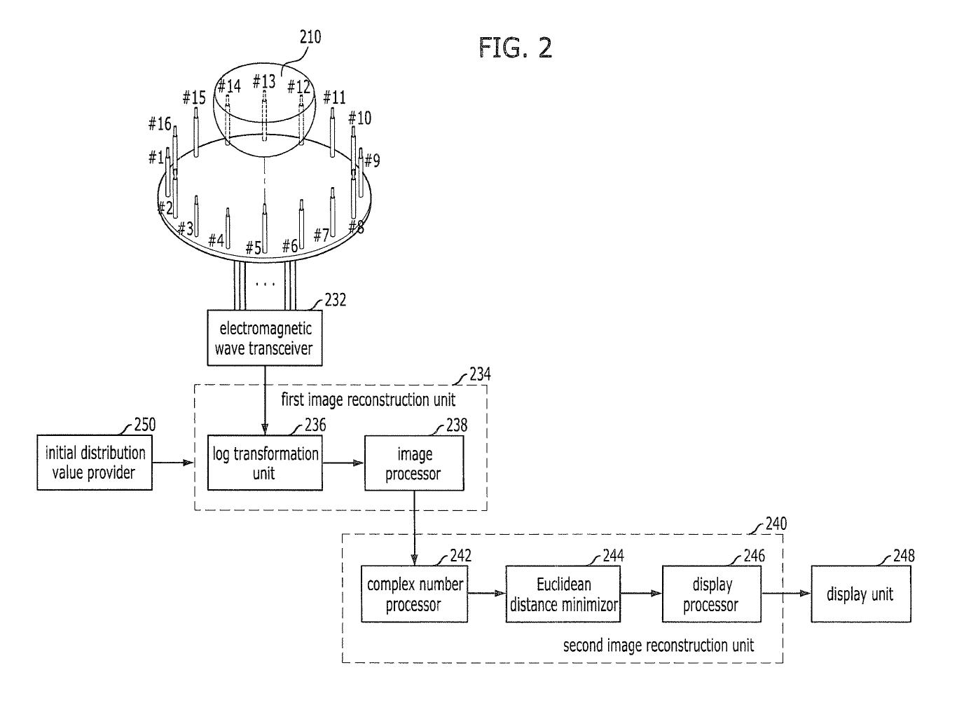

Hereinafter, a breast cancer diagnosis apparatus and method in accordance with an embodiment of the present invention will be described with reference to FIG. 2.

FIG. 2 is a perspective view of a breast cancer diagnosis apparatus in accordance with an embodiment of the present invention.

Referring to FIG. 2, the breast cancer diagnosis apparatus according to the present embodiment includes a tank (not shown) for providing a space to insert a breast 210 of a patient. The tank is filled with a predetermined liquid having a dielectric constant similar to that of a breast. The tank may be configured of a free space without the predetermined liquid filed with. The tank may have a size enough to house a target object for diagnosing a cancer, for example, a breast 210. It is preferable that the tank has a circular shape. However, the present invention is not limited thereto. That is, the tank may be formed in various shapes such as a rectangular shape and a polygonal shape.

As shown in FIG. 2, the tank includes predetermined devices. The tank includes a plurality of antennas #1, #2, . . . , and #16. The plurality of antennas #1, #2, . . . , and #16 receive and transmit electromagnetic waves. Each of the antennas may expand or contract within a predetermined range. That is, the length of each antenna #1, #2, . . . , and #16 may be changed within a predetermined range. Further, the number of a plurality of antennas #1, #2, . . . , and #16 is not limited to 16 as shown in FIG. 2. According to the design selection of a user or a designer, the tank may include more antennas or less antennas than 16 antennas. In the present embodiment, the breast cancer diagnosis apparatus includes 16 antennas and a tank having a circular shape.

When one of the plurality of antennas #1, #2, . . . , and #16 radiates an electromagnetic wave, the other antennas receive the radiated electromagnetic wave. The plurality of antennas #1, #2, . . . , and #16 are disposed at one side of a circular tank in a circular form. Also, the plurality of antennas #1, #2, . . . , and #16 are controlled by a processor (not shown). That is, the processor (not shown) controls transmitting and receiving an electromagnetic wave or expanding and contracting an antenna within a predetermined length range.

When a patient puts a breast 210 having a tumor tissue in the tank, the plurality of antennas #1, #2, . . . , and #16 are controlled to have an initial length by expanding or contracting the antennas. After setting the initial length, the first antenna #1 is assigned as a transmitting antenna and the other antennas #2 to #16 are set as receiving antennas under control of the processor. The tank has an enough size to house a target object to diagnose, for example a breast 210.

For example, the processor may expand the transmitting antenna #1 and the receiving antennas #2 to #16 to have the maximum length in consideration of a shape of a breast 210 at an initial scanning stage. In this case, the processor controls the plurality of antennas to radiate an electromagnetic wave having a predetermined frequency with a predetermined intensity while gradually reducing the lengths of the plurality of antennas in a direction from an edge of breast to a nipple. Here, the edge of the breast is a part where the breast is closest to a chest. On the contrary, the processor may control the plurality of antennas to radiate an electromagnetic wave having a predetermined frequency with a predetermined intensity while gradually expanding the lengths of the plurality of antennas in a direction from a nipple to an edge of a breast. Here, the edge of the breast is a part where the breast is closest to a chest. The intensity of the radiated electromagnetic wave may be determined in consideration of a spacious state of a breast 210 and a distance between a transmitting antenna and a receiving antenna.

Hereinafter, a breast cancer diagnosing procedure will be described in more detail. The plurality of antennas #1, #2, . . . , and #16 extend maximally, the first antenna #1 is set as the transmitting antenna, and the second to sixteenth antennas #2 to #16 are set as the receiving antennas. Then, each of the receiving antennas #2, . . . , and #16 may measure an intensity and a phase angle of a signal transmitted from the first antenna #1. Then, the second antenna #2 is set as a transmitting antenna after setting all of the antennas to have the same length, and the second antenna #2 radiates an electromagnetic wave with predetermined signal intensity. Then, the other receiving antennas #1, #3, . . . , and #16 are controlled to operate as receiving antennas except the second antenna #2. By this procedure, each of the antennas #1 to #16 operates as the transmitting antenna at least once at a position where all of the antennas have the same length.

After the process controls all of antennas as the transmitting antenna at least once as described above, the lengths of antennas are reduced to a next predetermined length. After reducing the lengths of antennas, the above described procedure of transmitting and receiving electromagnetic waves repeats.

The processor collects information about amplitude and phase of the received electromagnetic wave corresponding to a radiation pattern of all antennas #1, #3, . . . , #16 at each position during the above electromagnetic wave transmitting and receiving procedures.

As shown in FIG. 2, the breast cancer diagnosis apparatus according to the present embodiment further includes an electromagnetic wave transceiver 232, an initial distribution value provider 250, a first image reconstruction unit 234, a second image reconstruction unit 240, and a display unit 248. The first image reconstruction unit 234 includes a log transform unit 236 and an image processor 238. The second image reconstruction unit 240 includes a complex number processor 242, a Euclidean distance minimizor 244, and a display processor 246. The initial distribution value provider 250 sets an initial dielectric constant and conductivity distribution value of a breast of a patient. Since the dielectric contact and conductivity distribution value is slightly different according to an age of a patient, a size of a breast, and a shape of a breast, an examiner such as a medical doctor may voluntary select an initial dielectric constant and conductivity distribution value. Since the dielectric contact and conductivity values of constituent material of a breast are well known, an initial dielectric constant and conductivity distribution value can be set very close to real dielectric constant and conductivity distribution value of a breast.

The electromagnetic wave transceiver 232 receives information about the amplitudes and the phases of received electromagnetic waves from the plurality of antennas #1-#16 through sixteen lines. The information about the amplitudes and the phases of the received electromagnetic waves are necessary data to reconstruct an internal image of a breast. The electromagnetic wave transceiver 232 transfers the received amplitude and phase information to a first image reconstruction unit 234. The first image reconstruction unit 234 includes a log transform unit 236 and an image processor 238. The log transform unit 236 receives the amplitude information of the received electromagnetic wave from the electromagnetic wave transceiver 232 and transforms the received amplitude information through a Log Transform algorithm. The Log Transform algorithm can improve sensitivity. Therefore, the first image reconstruction unit 234 can obtain a valid initial estimated image value with high contrast through only few calculation processes by applying the Log Transform algorithm for an electric field value.

Such an operation of the first image reconstruction unit 236 will be described in more detail hereinafter. For example, in a graph of “y=ax” where a is a constant, a distance between two points (x1, y1) and (x2, y2) has a straight line value. If the amplitude information is transformed using the Log Transform algorithm, a Log Scale graph is obtained. In the Log Scale graph, the distance between the same two points (x1, y1) and (x2, y2) is longer than that in the graph of “y=ax”. Since the disparity of a distance between given two points is increased by performing the Log Transform, the sensitivity can be improved.

The image processor 238 may obtain initial estimated image information by processing electric field values using a Levenberg-Marquardt (LM) algorithm or a Tikhonov (TK) algorithm. In general, the LM and TK algorithms may be expressed as Eq. 1 based on a least square method.

P=min|E^{m}−E^{c}(k²)|² Eq. 1

A parameter E^{m} is a value measured by an image reconstruction apparatus, and a parameter E^{m} denotes a calculated electric field value. It is necessary to accurately and quickly find a value of k for image reconstruction.

The first image reconstruction unit 234 may obtain a valid initial estimated image value through few calculation steps. Such a first image reconstruction step can be expressed as Eq. 2.

P′=min∥Γ^{m}−Γ^{e}(k²)∥²+∥Φ^{m}−Φ^{e}(k²)∥² Eq. 2

The minimum k value obtained from Eq. 2 is used as k_{IM} in the second image reconstruction unit 240.

In Eq. 2, a parameter Γ is a result of Log Transform of an amplitude value of the measured electric field value E^{m} and the amplitude value of the calculated electric field value E^{c}. That is, Γ^{m}=log|E^{m}.

In Eq. 2, a parameter Φ denotes a phase of the measured electric field value E^{m} and a phase of the calculated electric field value E^{c}. Therefore, Φ^{m}=∠E^{m2}.

However, the first image reconstruction unit 230 may infinitely repeat the calculation. In order to prevent this problem, the breast cancer diagnosis apparatus according to the present embodiment includes the second image reconstruction unit 240 for stable image reconstruction.

As shown in FIG. 2, the second image reconstruction unit 240 includes a complex number processor 242, a Euclidean distance minimizor 244, and a display processor 246.

The complex number processor 242 expresses the initial estimated image value outputted from the image processor 238 of the first image reconstruction unit 234 in a complex number form. In general, the dielectric constant of material is expressed in a complex number form. A dielectric constant value means a real part of a dielectric constant, and conductivity means an imaginary part of a complex dielectric constant. A reconstructed image according to the present embodiment is expressed as distribution of dielectric constant values and conductivity values. Therefore, the breast cancer diagnosis apparatus according to the present embodiment uses a complex number form value including dielectric constant values and conductivity values which are the initial estimated image values. In the present embodiment, an image may be reconstructed by minimizing difference between the initial estimated image value and a new value estimated from the initial estimated image value. Here, both of the initial estimated image value and the new value are in a complex number form.

The first image reconstruction unit 234 uses the initial dielectric constant and conductivity distribution value, which is assigned by a user, from the initial distribution value provider 250. However, a second image reconstruction step uses the initial estimated image value obtained at the first image reconstruction unit 234. After the initial dielectric constant and conductivity distribution is set, electric fields of a target area to reconstruct an image are numerically calculated based on the set initial dielectric constant and conductivity distribution.

Then, a phase value and an intensity value of an electric field received at each of antennas disposed around a breast of a patient are extracted from the numerically calculated electric fields.

Then, the Euclidean distance minimizor 244 obtains differences in a phase and an intensity between an electric field E^{m} previously measured from a breast of a patient and the calculated electric field E^{c} using an Euclidean Distance Minimization algorithm as shown in Eq. 3.

P′=min∥E^{m}−E^{c}(k²)∥²+ρ∥k²−k_{IM}²∥² Eq. 3

In Eq. 3, a parameter k_{IM} denotes an output value obtained by the first image reconstruction unit 234 and p denotes an value inputted by a user such as an operator or a medical doctor.

By using the Euclidean Distance Minimization algorithm, the second image reconstruction unit 240 can reconstruct an image with higher sharpness than the initial estimation image obtained through the Log Transform algorithm in the first image reconstruction unit 234.

The information calculated and generated by the second image reconstruction unit 240 is provided to the display processor 246. The display processor 246 receives the generated information from the second image reconstruction unit 240, transforms the received information to graphic data, and provides the graphic data as reconstructed image to the display unit 248. The display unit 248 displays the reconstructed image from the display processor 246 through a monitor such as a CRT monitor or a LCD monitor. Accordingly, a medical doctor may accurately read a breast cancer in a breast of a patient based on the reconstructed image, such as a size and a location of a tumor tissue in a breast.

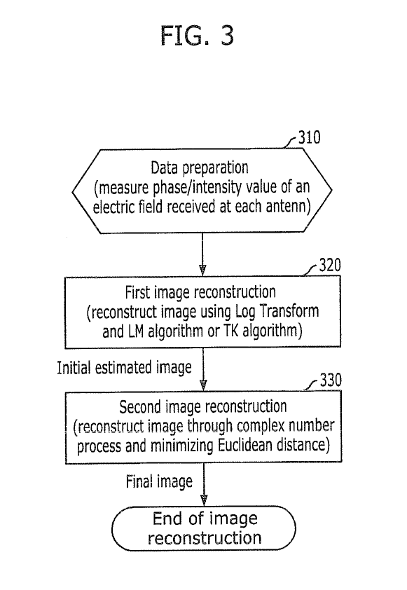

FIG. 3 is a flowchart illustrating two step microwave image reconstruction in accordance with another embodiment of the present invention.

At step S310, the electromagnetic wave transceiver 232 prepares measurement data to reconstruct an image by receiving information about an amplitude and a phase of an electromagnetic wave received through a plurality of antennas #1, #2, . . . , and #16.

At step S320, the first image reconstruction unit 234 obtains a valid initial estimated image value from the amplitude and phase information of the received electromagnetic wave from the electromagnetic wave transceiver 232 by transforming the amplitude values through Log Transform using the log transform unit 236. The image processor 238 obtains initial estimated image information by processing electric field values using a LM algorithm or a TK algorithm.

At step S320, the second image reconstruction unit 240 expresses the initial estimated image information outputted from the first image reconstruction unit 234 as a complex number form and uses a Euclidean Distance Minimization method.

Since the dielectric constant and conductivity of a material can be expressed in a complex number form, the complex number processor 242 expresses the initial estimated image value as values in a complex number form, and extracts a phase value and an intensity value of an electric field received from each of the antennas positioned around a breast of a patient from an numerically calculated electric field.

Then, differences in a phase and intensity between an electric field previously measured from a breast of a patient and the calculated electric field from the second image reconstruction unit 240 is minimized using a Euclidean distance minimization method.

The display processor 246 receives information generated from the second image reconstruction unit 240 to display the received information through a display device such as a monitor. That is, the display processor 246 receives the information generated from the second image reconstruction unit 240 and provides a reconstructed image by converting the received information to display them through the display unit 248 such as a monitor. A medical doctor may accurately determine a size or a location of adenocarcinoma existed in a breast of a patient from the provided image.

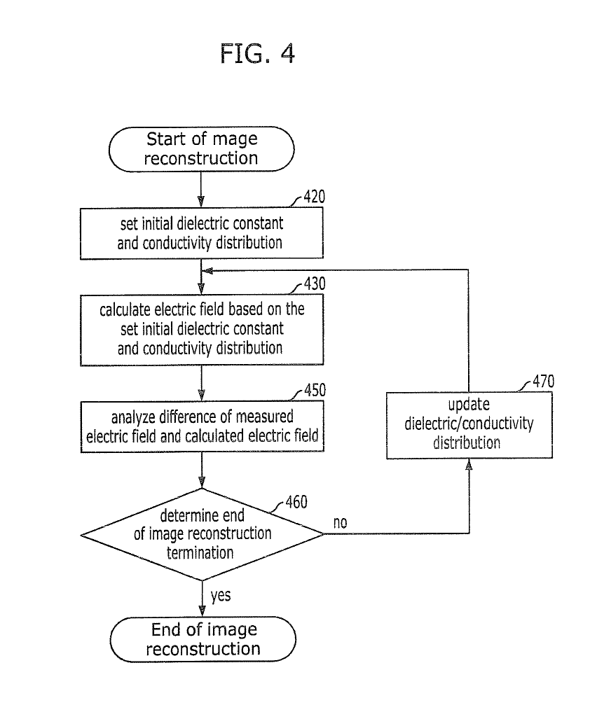

FIG. 4 is a flowchart illustrating an image reconstruction algorithm in accordance with an embodiment of the present invention.

At step S420, the initial distribution value provider 250 sets an initial dielectric constant and conductivity distribution value according to an age of a patient, a size of a breast, and a shape of a breast when image reconstruction starts.

At step S430, following operations are performed to numerically calculate an electric field for an image reconstruction area based on the set dielectric constant and conductivity.

The electromagnetic wave transceiver 232 receives amplitude and phase information of an electromagnetic wave received from a plurality of antennas #1, #2, . . . , and #16 and transfers the received amplitude and phase information to the first image reconstruction unit 234.

The log transformation unit 236 of the first image reconstruction unit 234 transforms amplitude values of the electromagnetic wave through Log Transformation to obtain a valid initial estimated image from the amplitude and phase information of the electromagnetic wave. The image processor 238 obtains initial estimated image information by processing electric field values using a Levenberg-Marquardt (LM) algorithm or a Tikhonov (TK) algorithm.

The second image reconstruction unit 240 numerically calculates an electric field of an area to reconstruct an image based on dielectric constant and conductivity, which are initial estimated image information obtained from the first image reconstruction unit 234. The complex number processor 242 of the second image reconstruction unit 240 expresses the initial estimated image, which is the output of the image processor 238 of the first image reconstruction unit 234, in a complex number form.

A phase and an intensity value of an electric field received from each of antennas positioned around a breast of a patient are extracted from the numerically calculated electric field. At step S450, the Euclidean distance minimizor 244 analyzes a difference in a phase and intensity between an electric field measured from a breast of a patient and the numerically calculated electric field using a Euclidean distance minimization method.

At step S460, the second image reconstruction unit 240 determines whether the analyzed difference in the phase and the intensity between the measured electric field and the calculated electric field is matched with a predetermined determination standard or not and determines whether the image reconstruction algorithm ends or not. If it determines that the image reconstruction algorithm does not end, the electromagnetic wave transceiver 232 receives amplitude and phase information of electromagnetic wave received through a plurality of antennas #1, #2, #3, . . . , and #16 again for updating dielectric constant and conductivity distribution at the step S470. The above steps are repeatedly performed until a predetermined termination condition is satisfied by numerically calculating an electric field based on a dielectric constant and conductivity value which is updated by performing the operations of the first image reconstruction unit 234 and the second image reconstruction unit 240.

If it determines that the image reconstruction algorithm ends, the information generated from the second image reconstruction unit 240 is inputted to the display processor 246 and converted into graphic data. The graphic data from the display processor 246 is displayed through the display unit 248 such as a monitor, thereby providing a restored image.

A medical doctor may accurately read a size and a location of adenocarcinoma existed in a breast of a patient from the displayed image on the display unit.

As described above, the breast cancer diagnosis apparatus and method according to the present embodiment can quickly obtain a valid initial estimated image value with only few calculation steps and can provide improved and stable image reconstruction effect using the initial estimated image value.

While the present invention has been described with respect to the specific embodiments, it will be apparent to those skilled in the art that various changes and modifications may be made without departing from the spirit and scope of the invention as defined in the following claims.

What is claimed is:

1. An apparatus for diagnosing a breast cancer using an electromagnetic wave, comprising:
a plurality of antennas disposed in a tank for inserting a breast and configured to transmit and receive the electromagnetic wave;
an electromagnetic wave transceiver configured to receive phase and amplitude information of the electromagnetic wave received through the plurality of antennas;
an initial distribution value provider configured to set an initial dielectric constant and conductivity distribution value for dielectric constant and conductivity of a breast of a patient and in the tank;
a first image reconstruction unit configured to transform the amplitude information of the electromagnetic wave through log transform and to obtain a first image by calculating a phase and amplitude information value of electric field generated from the electromagnetic wave; and
a second image reconstruction unit configured to transform the first image in a value in a complex number form and to transform values, which make a difference in a phase and an intensity between the measured electric field and the calculated electric field to have a minimum distance, to a second image.

2. The apparatus of claim 1, wherein the first image reconstruction unit includes:
a log transformation unit configured to transform amplitude information of the electromagnetic wave through log transformation; and
an image processor configured to transform the phase and amplitude information of the electric field to the first image information through a predetermined image algorithm.

3. The apparatus of claim 2, wherein the image algorithm is a Levenberg-Marquardt (LM) algorithm.

4. The apparatus of claim 2, wherein the image algorithm is a Tikhonov (TK) algorithm.

5. The apparatus of claim 1, wherein the second image reconstruction unit includes:
a complex number processor configured to transform the obtained first image information to values in a complex number form; and
an Euclidean distance minimization unit configured to detect a difference in a phase and an intensity between an electric field measured at the electromagnetic wave transceiver and the calculated electric field and to generate the second image information by obtaining values that minimize an Euclidean distance.

6. The apparatus of claim 1, further comprising a display unit configured to display the second image.

7. A method for diagnosing a breast cancer using an electromagnetic wave, comprising:
inserting a breast in a tank and transmitting and receiving an electromagnetic wave using a plurality of antennas disposed in the tank;
setting an initial dielectric constant and conductivity distribution value for dielectric constant of predetermined liquid and conductivity of the breast and in the tank;
transforming amplitude information of the electromagnetic wave through Log Transformation and obtaining first image information by calculating a phase and amplitude information value of electric field generated from the electromagnetic wave through a predetermined image algorithm; and
transforming the obtained first image information to values in a complex number form, and transforming values that makes a difference in a phase and an intensity between measured electric field from the antennas and the calculated electric field to have a minimum Euclidean distance to a second image.

8. The method of claim 7, wherein the predetermined image algorithm is a Levenberg-Marquardt (LM) algorithm.

9. The method of claim 7, wherein the predetermined image algorithm is a Tikhonov (TK) algorithm.

10. The method of claim 7, wherein said transforming the obtained second image information to values includes:
transforming the obtained first image information into values in a complex number form; and
detecting phase and intensity differences between an electric field measured from the plurality of antennas and the calculated electric field and generating the second image information and obtaining values that minimize an Euclidean distance.

11. The method of claim 7, further comprising:
displaying the second image.

12. A method for diagnosing a breast cancer using an electromagnetic wave, comprising:
setting an initial dielectric constant and conductivity distribution value for dielectric constant and conductivity of a breast of a patient;
obtaining phase and amplitude information of electric field generated from the electromagnetic wave received from a plurality of extractable antennas that transmit and receive the electromagnetic wave, transforming the obtained amplitude information of the electromagnetic wave through Log Transform, and obtaining estimated image information by processing the phase and amplitude information of the electric field through a predetermined image algorithm;
obtaining a phase value and an intensity value of an electric field by transforming the obtained estimated image information into values in a complex number form;
detecting a difference between a phase value and an intensity value of an electric field measured from a breast of a patient and the obtained phase value and intensity value of the electric field for said obtaining a phase value and intensity value; and
providing the image information when the detected error is in a predetermined range.

13. The method of claim 12, further comprising repeating said obtaining phase and amplitude information of an electromagnetic wave to said providing the image information when the detected difference exceeds the predetermined range.
